(19) United States
(12) Patent Application Publication
(10) Pub. No.: US 2007/0048379 A1
(43) Pub. Date: Mar. 1, 2007

(54) Valproate tablet

(76) Inventors: Mintong Guo, Plainsboro, NJ (US); Han-Pin Lim, Cranbury, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/214,135

(22) Filed: Aug. 29, 2005

(51) Int. Cl. A61K 9/28; A61K 9/24

(52) U.S. Cl. 424/472; 427/2.14

(57) ABSTRACT

A process for preparing a pharmaceutical tablet comprising: (a) mixing a valproate compound with at least one excipient to form a premix; (b) adding a sufficient amount of water to the premix of Step (a) and mixing to form a wet granulation; (c) drying the wet granulation of Step (b) at a temperature and time sufficient to form granules, wherein the granules are essentially free of an organic solvent; (d) coating the granules of Step (c) to form coated granules; and (e) compressing the coated granules of Step (d) to form a tablet. The aqueous granulation of the invention does not have the environmental and safety drawbacks of organic solvent granulations.

### FIELD OF THE INVENTION

The invention relates to a process for preparing a tablet comprising divalproex sodium.

### BACKGROUND OF THE INVENTION

Valproic acid and its derivative, divalproex, are oral drugs that are used for the treatment of seizures, bipolar disorder and prevention of migraines. The active ingredient in both products is valproic acid or valproate. Valproate acid has the formula: 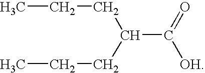
Scientists do not know the mechanism of action of valproate. The most popular theory is that valproate exerts its effects by increasing the concentration of gamma-aminobutyric acid (GABA) in the brain. GABA is a neurotransmitter, a chemical that nerves use to communicate with one another.

Divalproex is commercially-available from Abbott as Depakote®. The following forms are available: Depakote® delayed-release tablets: 125, 250 and 500 mg; Depakote® sprinkle capsules: 125 mg; Depakote® ER tablets: 500 mg; Depakote® capsules: 250 mg; Depakote® syrup: 250 mg/5 mL; Depacon® (valproate sodium) injection: 100 mg/5 mL; Valproic acid capsules: 250 mg; and Valproic acid syrup: 250 mg/5 mL.

U.S. Pat. No. 5,019,398 describes a sustained release tablet of valproic acid wherein the valproate is granulated with an organic solvent, such as alcohols, ketones or halogenated hydrocarbons. The most preferred granulating solvent is ethanol. U.S. Pat. No. 5,598,191 describes the preparation of sodium valproate tablets. Examples 1 and 3-6 utilize a granulation step. Ethanol is used as the granulating solvent in these examples. U.S. Pat. No. 5,268,182 describes the preparation of sustained release tablets containing divalproex sodium. Examples 1 and 2 describe the preparation of a divalproex sodium dosage form in which ethanol was used as the granulation solvent. U.S. Pat. No. 5,049,586 describes the preparation of moisture stable valproic tablets. In Example 1, ethanol is used as the granulation solvent.

Patent Application Publication No. US 2002/0127277 describes solid dosage forms of divalproex sodium which is prepared by a wet granulation process using a low amount of an organic solvent. Residual amounts of these organic solvents are retained in the granulate despite the attempts to remove such solvents during the drying step.

Organic solvents, such as methylene chloride have negative consequences on the environment. Special precautions must be taken in their disposal to avoid environmental contamination. Further, organic solvents can present safety hazards to the employees during the manufacturing of such formulations. These solvents are explosive and flammable. Incidents have been reported in which workers have been injured from accidental fires. Organic solvents and alcohols are considerably more expensive than water. This plus the difficulty of disposal adds cost to the production process and ultimately to the consumer of the medication. Further, it is impossible to completely remove all traces of the organic solvent or alcohol from the granulated drug substance. Trace solvents can leave adverse odors in the finished dosage form that are objectionable to many patients.

Thus, the prior art teaches that organic solvents must be used in a granulation process involving valproic acid and other valproate compounds. There exists a need for a granulation process that may be used to produce tablets containing valproate compounds that does not require an organic solvent.

### SUMMARY OF THE INVENTION

The invention provides a pharmaceutical tablet comprising granules which comprise a valproate compound and at least one excipient, wherein said granules are essentially free of an organic solvent.

According to another aspect, the invention provides a pharmaceutical tablet comprising granules which comprise a valproate compound and at least one excipient, wherein said granules are coated with a film-forming polymer, and said granules are essentially free of an organic solvent.

According to another aspect, the invention provides a process for preparing a pharmaceutical tablet comprising:
(a) mixing a valproate compound with at least one excipient to form a premix;
(b) adding a sufficient amount of water to the premix of Step (a) and mixing to form a wet granulation;
(c) drying the wet granulation of Step (b) at a temperature and time sufficient to form granules, wherein the granules are essentially free of an organic solvent;
(d) coating the granules of Step (c) to form coated granules; and
(e) compressing the coated granules of Step (d) to form a tablet.

According to another aspect, the invention provides a process for preparing a pharmaceutical tablet comprising:
(i) mixing a valproate compound with at least one excipient to form a premix;
(ii) adding a sufficient amount of water and a film-forming polymer to the premix of Step (i) and mixing to form a wet granulation;
(iii) drying the wet granulation of Step (ii) at a temperature and time sufficient to form granules, wherein the granules are essentially free of an organic solvent; and
(iv) compressing the granules of Step (iii) to form a tablet.

The aqueous granulation of the invention has numerous advantages when compared with the organic solvent granulations of the prior art. The process of the invention does not have the environmental and safety drawbacks of the prior art. Further, costs are significantly reduced with the process of the invention. Moreover, prior art granulations which are conducted with organic solvents inherently result in residual amounts of the organic solvent being retained in the granules. An additional advantage of the present invention is that even though water is used during wet granulation, sticking of the tablet ingredients on the tablet punches is reduced or eliminated.

### DESCRIPTION OF THE INVENTION

The invention provides a pharmaceutical tablet comprising granules which comprise a valproate compound and at least one excipient, wherein said granules are essentially free of an organic solvent. The invention also provides a pharmaceutical tablet comprising granules which comprise a valproate compound and at least one excipient, wherein said granules are coated with a film-forming polymer, and said granules are essentially free of an organic solvent.

In one embodiment of the invention, the tablets are prepared by Process I which comprises:
(a) mixing a valproate compound with at least one excipient to form a premix;
(b) adding a sufficient amount of water to the premix of Step (a) and mixing to form a wet granulation;
(c) drying the wet granulation of Step (b) at a temperature and time sufficient to form granules, wherein the granules are essentially free of an organic solvent;
(d) coating the granules of Step (c) to form coated granules; and
(e) compressing the coated granules of Step (d) to form a tablet.

In one embodiment of the invention, the tablets are prepared by Process II which comprises:
(i) mixing a valproate compound with at least one excipient to form a premix;
(ii) adding a sufficient amount of water and a film-forming polymer to the premix of Step (ii) and mixing to form a wet granulation;
(iii) drying the wet granulation of Step (ii) at a temperature and time sufficient to form granules, wherein the granules are essentially free of an organic solvent; and
(iv) compressing the granules of Step (iii) to form a tablet.

As used herein, “valproate compounds” includes valproic acid, the sodium salt of valproate, divalproex sodium, salts of valproate, prodrugs of valproate, analogs of valproate, amides of valproate, esters of valproate and pharmaceutically acceptable salts thereof, especially basic addition salts. As used herein, “valproate compounds” also includes compounds which readily metabolize in vivo to produce valproate, such as valproate amide (valproimide). Divalproex sodium is the most preferred valproate compound.

Analogs of valproate include structures represented by formula (I): 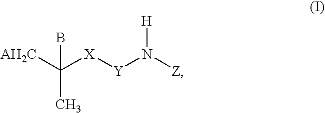
wherein

A=H, CH₃ or OH;

B=H, OH or CH₃;

X=CH₂, CH CH₃, C(CH₃)₂, —O—, CH(OH), or —CH₂O—;

Y=—CO— or —SO₂—; and

Z=H, CH₂CO₂H, or CH₂CONH₂.

A preferred analog of valproate is isovaleramide which is represented by formula (I),

wherein

A, B and Z are H;

Y is CO; and

X is CH₂.

Other analogs of valproate include structures represented by formula (II): 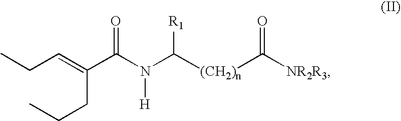
wherein

R₁, R₂ and R₃ are independently H, C₁-C₆alkyl, aralkyl or aryl; and

n is an integer which is greater than or equal to 0 and less than or equal to 3.

Further analogs of valproate have been prepared in which either unsaturation or a cycloalkyl moiety has been incorporated into one of the proply moieties of the valproate structure. Examples of such analogs include structures represented by formula (III):

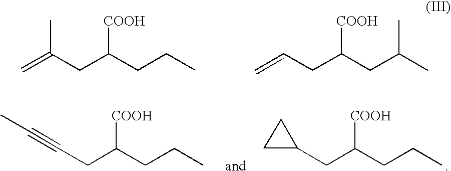

The valproate compound may optionally be subjected to milling or screening to reduce the particle size of the valproate compound. For example, small amounts can be forced by hand through a manual screen. Large quantities can be forced through sieving devices such as a Stokes oscillator, a Colton rotary granulator, a Stokes mill, a Fitzpatrick mill, etc. Preferably, the valproate compound has a particle size of less than 4000 microns, and more preferably less than 200 microns.

The valproate compound is present in an amount of from about 10 weight percent (wt. %) to about 80 wt. %, based on the total weight of the tablet. Preferably, the valproate compound is present in an amount of from about 40 wt. % to about 70 wt. %, more preferably from about 50 wt. % to about 60 wt. %, based on the total weight of the tablet. Typically, the amount of valproate compound in the tablets of the invention varies from about 100 mg to about 1000 mg, preferably from about 125 mg to about 500 mg, based on the amount of valproic acid.

In the first step of Process I and Process II, Step (a) and Step (i), a valproate compound is mixed with at least one excipient to form a premix. The valproate compound and excipient(s) are mixed together using techniques well-known in the art. Typically, they will be dry-blended together in a device, such as a high-shear mixer. Other suitable devices include V-blender, bin blender, etc.

Examples of excipients which may be used in the tablets of the invention include, but are not limited to, surfactants, diluents, binders, solubilizers, disintegrants, fillers, lubricants, buffers, stabilizers, colorants, dyes, anti-oxidants, anti-adherents, preservatives and glidants. A mixture of excipients may also be used.

In the second step of Process I, Step (b), a sufficient amount of water is added to the premix of Step (a) and mixed to form a wet granulation.

In the second step of Process II, Step (ii), a sufficient amount of water and a film-forming polymer is added to the premix of Step (i) and mixed to form a wet granulation.

The film-forming polymer is preferably selected from cross-linked polyvinyl pyrrolidone; non-cross-linked polyvinylpyrrolidone; hydroxypropylmethyl cellulose phthalate; hydroxypropylmethyl cellulose acetate succinate; cellulose acetate succinate; cellulose acetate phthalate; hydroxypropylmethyl cellulose acetate succinate; cellulose acetate trimellitate; hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene co-polymer; polyvinylalcohols; co-polymers of acrylic acid and/or methacrylic acid with at least one monomer selected from the group consisting of methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate and octadecyl acrylate; polyvinyl acetate; fats; oils; waxes; fatty alcohols; shellac; gluten; ethylacrylate-maleic acid anhydride co-polymer; maleic acid anhydride-vinyl methyl ether co-polymer; styrol-maleic acid co-polymer; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); polyurethane; polyvinyl/maleic anhydride co-polymers; poly(methacrylic acid); ethylene/maleic anhydride co-polymers; and ammonio methacrylate co-polymers. Ammonio methacrylate co-polymers comprise acrylic and/or methacrylic ester groups together with quaternary ammonium groups. A mixture of film-forming polymers may also be used. More preferably, the film-forming polymer is hydroxypropylmethyl cellulose.

In one embodiment of the invention, the film-forming polymer additionally comprises a plasticizer. The plasticizer is preferably selected from acetyl-triethyl citrate, acetyl tributyl-, tributyl-, triethyl-citrate, glycerol diacetate, glycerol triacetate, acetylated monoglycerides, castor oil, dibutyl-phthalate, diamyl-phthalate, diethyl-phthalate, dimethyl-phthalate, dipropyl-phthalate, di-(2-methoxy- or 2-ethoxyethyl)-phthalate, ethylphthalyl glycolate, butylphthalylethyl glycolate, butylglycolate, propylene glycol, polyethylene glycol, diethyladipate, di-(2-methoxy- or 2-ethoxyethyl)-adipate, benzophenone, diethyl- and diburylsebacate, dibutylsuccinate, dibutyltartrate, diethylene glycol dipropionate, ethyleneglycol diacetate, ethyleneglycol dibutyrate, ethyleneglycol dipropionate, tributyl phosphate, tributyrin, PEG sorbitan monooleate and sorbitan monooleate. A mixture of plasticizers may also be used.

The amount of the film-forming polymer used in Step (ii) of Process II, is preferably from about 1 wt. % to about 30 wt. %, based on the total weight of the premix of Step (i) which is used to form the wet granulation. More preferably, the amount of the film-forming polymer is from about 2 wt. % to about 10 wt. %, most preferably from about 3 wt. % to about 7 wt. %, based on the total weight of the premix.

In the third step of Process I, Step (c), and in the third step of Process II, Step (iii), the wet granulation prepared in Step (b) or Step (iii), respectively, is dried at a temperature and time sufficient to form granules. The granules are essentially free of an organic solvent.

As used herein, “essentially free of an organic solvent” means that the granules which are prepared after drying the wet granulation contain less than 0.1 wt. % of an organic solvent, preferably less than 0.05 wt. %, based on the weight of the granules. More preferably, the granules do not contain any organic solvent. Examples of organic solvents include, but are not limited to, ethanol, propanol, butanol, acetone, butanone, ethyl acetate and methylene chloride.

Drying techniques include fluid bed, flash drying, ring drying, micron drying, tray drying, vacuum drying, radio-frequency drying and microwave drying. Preferably, the granules are dried to a moisture content of 3 w/w % or less, more preferably 2 w/w % or less. Preferably, the wet granulation is dried in an oven at a temperature range of from about 40-80° C. for a period of time ranging from about 4 hours to about 15 hours, or dried in a fluid bed dryer in which the wet granulation is suspended and agitated in a warm air stream.

Once the drying is completed, the granules may optionally be subjected to milling or screening to reduce the particle size. Sieving devices include a Stokes oscillator, a Colton rotary granulator, a Stokes mill, a Fitzpatrick mill. etc. A particle size in the range of about 40 microns to about 1200 microns is preferred.

In the fourth step of Process I, Step (d), the dried granules prepared in Step (c) are coated to form coated granules. Optionally, in Process II, the dried granules prepared in Step (iii) may be coated to form coated granules. The coating comprises a film-forming polymer. The film-forming polymer is preferably selected from cross-linked polyvinyl pyrrolidone; non-cross-linked polyvinylpyrrolidone; hydroxypropylmethyl cellulose phthalate; hydroxypropylmethyl cellulose acetate succinate; cellulose acetate succinate; cellulose acetate phthalate; hydroxypropylmethyl cellulose acetate succinate; cellulose acetate trimellitate; hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene co-polymer; polyvinylalcohols; co-polymers of acrylic acid and/or methacrylic acid with at least one monomer selected from the group consisting of methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate and octadecyl acrylate; polyvinyl acetate; fats; oils; waxes; fatty alcohols; shellac; gluten; ethylacrylate-maleic acid anhydride co-polymer; maleic acid anhydride-vinyl methyl ether co-polymer; styrol-maleic acid co-polymer; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); polyurethane; polyvinyl/maleic anhydride co-polymers; poly(methacrylic acid); ethylene/maleic anhydride co-polymers; and ammonio methacrylate co-polymers. Ammonio methacrylate co-polymers comprise acrylic and/or methacrylic ester groups together with quaternary ammonium groups. A mixture of film-forming polymers may also be used.

More preferably, the film-forming polymer used in the coating to coat the granules is a water-soluble polymer having a viscosity of less than about 50 centipoise (cps), as determined using a Ubbelohde viscometer, 2% by weight of polymer in water, at a temperature of 20° C.±0.1° C., according to the American Society for Testing and Materials (ASTM, D445). More preferably, the water-soluble polymers have a viscosity of about 1 cps to about 30 cps, most preferably, from about 5 cps to about 20 cps.

The film-forming polymer used in the coating to coat the granules is more preferably selected from hydroxypropylmethyl cellulose, hydroxypropyl cellulose (HPC), hydroxyethyl cellulose, methyl cellulose, sodium carboxymethyl cellulose, hydroxyethylmethyl cellulose, ethylcarboxyethyl cellulose, polyvinylalcohol, sodium alginate, polyvinylpyrrolidone, vinyl acetate/crotonic acid copolymers, methyl methacrylic ester copolymers, maleic anhydride/methyl vinyl ether copolymers and poly(ethylene oxide). Most preferably, the film-forming polymer is hydroxypropylmethyl cellulose or hydroxypropyl cellulose.

Hydroxypropylmethyl cellulose is a polymer which is available in many forms, including forms of different molecular weight, extremely different viscosity and different substitution grade. It is within the scope of the invention to use mixtures or blends of two or more different forms of hydroxypropylmethyl cellulose as the water-soluble polymer in the coating. A preferred form of hydroxypropyl methylcellulose is that having a viscosity in the range 3-100 cps at 20° C. (U.S. National Formulary XIII), and more preferably a viscosity of 15 cps at 20° C. For example, Methocel E15 LV Premium, which has a viscosity of 15 cps, and is available from Dow Chemical.

HPC is a partially substituted poly(hydroxypropyl) ether of cellulose. HPC is commercially-available in a number of different grades which have different solution viscosities. The molecular weight of the HPC ranges from about 50,000 to about 1,250,000. A preferred HPC is available from Aqualon under the trademark KLUCEL. Suitable grades of HPC include the following:
1) KLUCEL EF having a molecular weight of about 80,000;
2) KLUCEL LF having a molecular weight of about 95,000;
3) KLUCEL JF having a molecular weight of about 140,000;
4) KLUCEL GF having a molecular weight of about 370,000;
5) KLUCEL MF having a molecular weight of about 850,000; and
6) KLUCEL HF having a molecular weight of about 1,150,000.

Included within the term “HPC” is a low-substituted hydroxypropyl cellulose (L-HPC). The L-HPC useful in the compositions of the invention is available in a number of different grades which have different particle sizes and substitution levels, and which are classified on the basis of their percent hydroxypropoxy content. When dried at 105° C. for 1 hour, the L-HPC contains from about 5% to about 16% of hydroxypropoxy groups, preferably from about 10% to about 13% of hydroxypropoxy groups. Suitable grades of L-HPC include the following:
1) LH-11 having a hydroxypropoxy content of 11% and an average particle size of 50 microns;
2) LH-21 having a hydroxypropoxy content of 11% and an average particle size of 40 microns;
3) LH-31 having a hydroxypropoxy content of 11% and an average particle size of 25 microns;
4) LH-22 having a hydroxypropoxy content of 8% and an average particle size of 40 microns;
5) LH-32 having a hydroxypropoxy content of 8% and an average particle size of 25 microns;
6) LH-20 having a hydroxypropoxy content of 13%, and an average particle size of 40 microns; and
7) LH-30 having a hydroxypropoxy content of 13%, and an average particle size of 25 microns.

Preferred L-HPCs are commercially-available from Shin-Etsu Chemical Company under the trade designation L-HPC Grade LH-21 and LH-11.

In one embodiment of the invention, the coating additionally comprises a plasticizer. The plasticizer is preferably selected from acetyl-triethyl citrate, acetyl tributyl-, tributyl-, triethyl-citrate, glycerol diacetate, glycerol triacetate, acetylated monoglycerides, castor oil, dibutyl-phthalate, diamyl-phthalate, diethyl-phthalate, dimethyl-phthalate, dipropyl-phthalate, di-(2-methoxy- or 2-ethoxyethyl)-phthalate, ethylphthalyl glycolate, butylphthalylethyl glycolate, butylglycolate, propylene glycol, polyethylene glycol, diethyladipate, di-(2-methoxy- or 2-ethoxyethyl)-adipate, benzophenone, diethyl- and diburylsebacate, dibutylsuccinate, dibutyltartrate, diethylene glycol dipropionate, ethyleneglycol diacetate, ethyleneglycol dibutyrate, ethyleneglycol dipropionate, tributyl phosphate, tributyrin, polyethylene glycol (PEG) sorbitan monooleate and sorbitan monooleate. A mixture of plasticizers may also be used.

The amount of plasticizer used in the coating which is used to coat the granules is preferably from about 1% to about 50%, more preferably from about 10% to about 25%, and most preferably about 20%, based on the weight of the film-forming polymer which is used in the coating.

A preferred coating for coating the granules is Opadry® which is available from Colorcon Corp. Opadry formulations may contain hydroxypropylmethyl cellulose, lactose, polydextrose, triacetin, polyethyleneglycol, polysorbate 80, titanium dioxide and one or more dyes.

The coating is preferably applied to the surface of the granules, preferably with a fluid bed coater. The amount of the coating is preferably from about 1 wt. % to about 30 wt. %, based on the total weight of the uncoated granules. More preferably, the amount of the coating is from about 2 wt. % to about 10 wt. %, most preferably from about 3 wt. % to about 7 wt. %, based on the total weight of the uncoated granules.

In the fifth step of Process I, Step (e), and in the fourth step of Process II, Step (iv), the granules are compressed to form a tablet. Tablet formulation and conventional processing techniques have been widely-described, e.g., “Pharmaceutical Dosage Forms: Tablets”, Lieberman, Ed., Lachman and Schwartz, Marcel Dekker, Inc., 2^{nd} Edition (1989), the text of which is herein incorporated by reference.

Optionally, the tablet is coated. The tablet coating comprises a film-forming polymer. Tablets may be coated to improve their appearance, protect them from atmospheric degradation, control the site of drug release (i.e. enteric coatings), delay or prolong their release patterns, etc. Methods for coating tablets are well-known in the art. The quantity of such coating can vary widely depending upon the reason for the coating. One skilled in the art can readily determine how much coating should be applied to the tablets in order to produce a desired result.

The film-forming polymer used to coat the tablet is preferably insoluble in gastric juice and soluble in intestinal juice at a pH >5. The film-forming polymer is preferably selected from cross-linked polyvinyl pyrrolidone; non-cross-linked polyvinylpyrrolidone; hydroxypropylmethyl cellulose phthalate; hydroxypropylmethyl cellulose acetate succinate; cellulose acetate succinate; cellulose acetate phthalate; hydroxypropylmethyl cellulose acetate succinate; cellulose acetate trimellitate; hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene co-polymer; polyvinylalcohols; co-polymers of acrylic acid and/or methacrylic acid with at least one monomer selected from the group consisting of methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate and octadecyl acrylate; polyvinyl acetate; fats; oils; waxes; fatty alcohols; shellac; gluten; ethylacrylate-maleic acid anhydride co-polymer; maleic acid anhydride-vinyl methyl ether co-polymer; styrol-maleic acid co-polymer; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); polyurethane; polyvinyl/maleic anhydride co-polymers; poly(methacrylic acid); ethylene/maleic anhydride co-polymers; and ammonio methacrylate co-polymers. Ammonio methacrylate co-polymers comprise acrylic and/or methacrylic ester groups together with quaternary ammonium groups. A mixture of film-forming polymers may also be used. More preferably, the film-forming polymer for coating the tablet is a polymer of methacrylic acid and an acrylic or methacrylic ester, such as methylacrylate.

In one embodiment of the invention, the film-forming polymer additionally comprises a plasticizer. The plasticizer is preferably selected from acetyl-triethyl citrate, acetyl tributyl-, tributyl-, triethyl-citrate, glycerol diacetate, glycerol triacetate, acetylated monoglycerides, castor oil, dibutyl-phthalate, diamyl-phthalate, diethyl-phthalate, dimethyl-phthalate, dipropyl-phthalate, di-(2-methoxy- or 2-ethoxyethyl)-phthalate, ethylphthalyl glycolate, butylphthalylethyl glycolate, butylglycolate, propylene glycol, polyethylene glycol, diethyladipate, di-(2-methoxy- or 2-ethoxyethyl)-adipate, benzophenone, diethyl- and diburylsebacate, dibutylsuccinate, dibutyltartrate, diethylene glycol dipropionate, ethyleneglycol diacetate, ethyleneglycol dibutyrate, ethyleneglycol dipropionate, tributyl phosphate, tributyrin, PEG sorbitan monooleate and sorbitan monooleate. A mixture of plasticizers may also be used.

The amount of plasticizer used with the film-forming polymer used to coat the tablet is preferably from about 5% to about 50%, more preferably from about 10% to about 25%, and most preferably about 20%, based on the weight of the film-forming polymer used to coat the tablet.

The film-forming polymer used to coat the tablet may be applied to the surface of the tablet. The amount of the film-forming polymer is preferably from about 1 wt. % to about 30 wt. %, based on the total weight of the uncoated tablet. More preferably, the amount of the film-forming polymer is from about 3 wt. % to about 20 wt. %, most preferably from about 5 wt. % to about 10 wt. %, based on the total weight of the uncoated tablet.

It is within the scope of the invention to introduce excipients in any of the process steps of the invention. Typically excipients are introduced via dry blending. Suitable dry blending devices include V-blenders, bin blenders, etc.

Examples of excipients which may be used in the tablets of the invention include, but are not limited to, surfactants, diluents, binders, solubilizers, disintegrants, fillers, lubricants, buffers, stabilizers, colorants, dyes, anti-oxidants, anti-adherents, preservatives and glidants. A mixture of excipients may also be used. Such excipients are known to those skilled in the art, and thus, only a limited number will be specifically referenced.

Examples of fillers or diluents include powdered sugar, calcium phosphate, calcium sulfate, microcrystalline cellulose, lactose, mannitol, kaolin, sodium chloride, dry starch, sorbitol, inositol, dibasic calcium phosphate dihydrate, calcium sulfate trihydrate and calcium sulfate dehydrate. A mixture of fillers may also be used. A preferred filler is microcrystalline cellulose. A preferred microcrystalline cellulose is, e.g., Avicel-PH-102 which has a nominal mean particle size of 90 microns, and is available from FMC Corporation. A filler or dilluent is preferably present in an amount of from about 1-70 wt. %, more preferably about 20 wt. % to about 60 wt. %, based on the weight of solid dosage form.

Examples of lubricants include talc, magnesium stearate, sodium stearate, calcium stearate, zinc stearate, stearic acid, vegetable oil, propylene glycol, PEG, stearic acid, sodium benzoate, sodium lauryl sulfate, magnesium lauryl sulfate, mineral oil and polyoxyethylene monostearate. A mixture of lubricants may also be used. A preferred lubricant is talc. A lubricant is preferably present in an amount of from about 0.1-10 wt. %, more preferably about 0.5 wt. % to about 5 wt. %, based on the weight of the solid dosage form.

Examples of binders include povidone, polyvinylpyrrolidone, xanthan gum, cellulose gums such as carboxymethylcellulose, methyl cellulose, hydroxypropylmethylcellulose, hydroxycellulose, gelatin, starch, pregelatinized starch, and microcrystalline cellulose such as products known under the registered trademarks Avicel, Filtrak, Heweten or Pharmacel. A mixture of binders may also be used. A binder is preferably present in an amount of from about 0-15 wt. %, more preferably about 5 wt. % to about 10 wt. %, based on the weight of the solid dosage form.

Examples of glidants include talc, silicon dioxide, cornstarch, silica, magnesium trisilicate, powdered cellulose, calcium silicate, and tribasic calcium phosphate. Colloidal silica, e.g., Aerosil, is particularly preferred. A mixture of glidants may also be used. A glidant is preferably present in an amount of from about 0-15 wt. %, more preferably about 3 wt. % to about 10 wt. %, based on the weight of the solid dosage form.

Examples of disintegrants include:
(i) cross-linked polyvinylpyrrolidones, e.g., crospovidones, such as Polyplasdone® XL and Kollidon® CL;
(ii) alginic acid and sodium alginate;
(iii) methacrylic acid-divinylbenzene co-polymer salts, e.g., Amberlite® IRP-88; and
(iv) cross-linked sodium carboxymethylcellulose, available as, e.g., Ac-di-sol®, Primellose®, Pharmacel® XL, Explocele and Nymcel® ZSX.
   Additional disintegrants also include hydroxypropylmethyl cellulose, croscarmellose sodium, polacrillin potassium, polyacrylates, such as Carbopol®, magnesium aluminium silicate, bentonite, sodium starch glycolate, pregelatinized corn starch, agar, and guar gum. A disintegrant is preferably present in an amount of from about 0-30 wt. %, more preferably about 5 wt. % to about 25 wt. %, based on the weight of the solid dosage form.

The following non-limiting examples illustrate further aspects of the invention.

### EXAMPLE 1

Preparation of Tablets Containing Divalproex Sodium.

| Ingredients | Amount | |
|---|---|---|
| Divalproex Sodium | 1724.29 | g |
| Microcrystalline Cellulose | 677.31 | g |
| (Avicel pH 102) | | |
| Sodium Starch Glycolate | 152.0 | g |
| Opadry White | 50.0 | g |
| Microcrystalline Cellulose | 115.25 | g |
| (Avicel pH 102) | | |
| Syloid 244 FP | 23.05 | g |
| Magnesium Stearate | 46.09 | g |
| Total | 2787.99 | |

Divalproex sodium, 1724.29 g, is charged along with 677.31 g of microcrystalline cellulose, and 152 g of sodium starch glycolate into a Glatt VG-10 high-shear granulator. The material is dry mixed for 20 minutes at low speed. The material is wet granulated with 150 g of water and mixed at a chopper speed of 2500 rpm and an impeller speed of 500 rpm. The material is dried in an oven at 40° C. to a loss on drying (LOD) of not more than 2%. The dried granulation is milled using a Fitzmill at medium speed with a screen size of #40.

A coating solution is prepared by dispersing 50 g of Opadry White in 450 g of water. The resulting granules are coated using a Glatt GPCG-1 fluid bed with a Wurster column.

The coated granules are collected and sifted through a 20-mesh screen, then mixed with 115.25 g of microcrystalline cellulose (Avicel pH 102), 23.05 g of Sylloid 244FP (silicon dioxide), and 46.09 g of magnesium stearate in a V-blender. The resulting blend is compressed on a tablet press to form tablets weighing 850-900 mg.

No sticking of the ingredients was observed on the tablet punches.

### EXAMPLE 2 (COMPARATIVE)

Preparation of Tablets Containing Divalproex Sodium.

| Ingredients | Amount | |
|---|---|---|
| Divalproex Sodium | 1077.7 | g |
| Microcrystalline Cellulose | 432.8 | g |
| (Avicel pH 101) | | |
| Sodium Starch Glycolate | 190.0 | g |
| Pregelatinized Starch (Starch 1500) | 95.0 | g |
| Povidone K-30 | 47.5 | g |
| Syloid 244 FP | 35.47 | g |
| Magnesium Stearate | 17.7 | g |
| Total | 1896.17 | g |

Divalproex sodium is charged along with microcrystalline cellulose, Starch 1500 and sodium starch glycolate into a Glatt VG-10 high-shear granulator. The material is dry mixed for 20 minutes at low speed. The material is wet granulated with 47.5 g of Povidone K-30 (polyvinylpyrrolidone) in 100 g of water and mixed at a chopper speed of 2500 rpm and an impeller speed of 500 rpm. The material is dried in an oven at 40° C. to a LOD of not more than 2%. The dried granulation is milled using a Fitzmill at medium speed with a screen size of #40.

The milled granules are mixed with 35.47 g of Sylloid 244FP, and 17.7 g of magnesium stearate in a V-blender. The resulting blend is compressed on a tablet press to form tablets. Sticking of the ingredients was observed on the tablet punches.

While the invention has been described with particular reference to certain embodiments thereof, it will be understood that changes and modifications may be made by those of ordinary skill within the scope and spirit of the following claims:

What is claimed is:

1. A process for preparing a pharmaceutical tablet comprising:
(a) mixing a valproate compound with at least one excipient to form a premix;
(b) adding a sufficient amount of water to the premix of Step (a) and mixing to form a wet granulation;
(c) drying the wet granulation of Step (b) at a temperature and time sufficient to form granules, wherein the granules are essentially free of an organic solvent;
(d) coating the granules of Step (c) to form coated granules; and
(e) compressing the coated granules of Step (d) to form a tablet.

2. A process for preparing a pharmaceutical tablet comprising:
(i) mixing a valproate compound with at least one excipient to form a premix;
(ii) adding a sufficient amount of water and a film-forming polymer to the premix of Step (i) and mixing to form a wet granulation;
(iii) drying the wet granulation of Step (ii) at a temperature and time sufficient to form granules, wherein the granules are essentially free of an organic solvent; and
(iv) compressing the granules of Step (iii) to form a tablet.

3. The process according to claim 1, which additionally comprises Step (f) coating the tablet of Step (e).

4. The process according to claim 2, which additionally comprises Step (v) coating the tablet of Step (iv).

5. The process according to claim 1, wherein the valproate compound is selected from the group consisting of divalproex sodium, valproic acid and mixtures thereof.

6. The process according to claim 1, wherein the valproate compound is present in an amount of from about 10 wt. % to about 80 wt. %, based on the total weight of the tablet.

7. The process according to claim 6, wherein the valproate compound is present in an amount of from about 40 wt. % to about 70 wt. %, based on the total weight of the tablet.

8. The process according to claim 2, wherein the film-forming polymer is selected from the group consisting of cross-linked polyvinyl pyrrolidone; non-cross-linked polyvinylpyrrolidone; hydroxypropylmethyl cellulose phthalate; hydroxypropylmethyl cellulose acetate succinate; cellulose acetate succinate; cellulose acetate phthalate; hydroxypropylmethyl cellulose acetate succinate; cellulose acetate trimellitate; hydroxypropyl methyl cellulose phthalate; hydroxypropyl methyl cellulose acetate succinate; starch acetate phthalate; polyvinyl acetate phthalate; carboxymethyl cellulose; methyl cellulose phthalate; methyl cellulose succinate; methyl cellulose phthalate succinate; methyl cellulose phthalic acid half ester; ethyl cellulose succinate; carboxymethylamide; potassium methacrylatedivinylbenzene co-polymer; polyvinylalcohols; co-polymers of acrylic acid and/or methacrylic acid with at least one monomer selected from the group consisting of methyl methacrylate, ethyl methacrylate, ethyl acrylate, butyl methacrylate, hexyl methacrylate, decyl methacrylate, lauryl methacrylate, phenyl methacrylate, methyl acrylate, isopropyl acrylate, isobutyl acrylate and octadecyl acrylate; polyvinyl acetate; fats; oils; waxes; fatty alcohols; shellac; gluten; ethylacrylate-maleic acid anhydride co-polymer; maleic acid anhydride-vinyl methyl ether co-polymer; styrol-maleic acid co-polymer; 2-ethyl-hexyl-acrylate maleic acid anhydride; crotonic acid-vinyl acetate co-polymer; glutaminic acid/glutamic acid ester co-polymer; carboxymethylethylcellulose glycerol monooctanoate; polyarginine; poly(ethylene); poly(propylene); poly(ethylene oxide); poly(ethylene terephthalate); poly(vinyl isobutyl ether); poly(vinyl chloride); polyurethane; polyvinyl/maleic anhydride co-polymers; poly(methacrylic acid); ethylene/maleic anhydride co-polymers; and ammonio methacrylate co-polymers, and mixtures thereof.

9. The process according to claim 8, wherein the film-forming polymer is selected from the group consisting of hydroxypropylmethyl cellulose, hydroxypropyl cellulose, hydroxyethyl cellulose, methyl cellulose, sodium carboxymethyl cellulose, hydroxyethylmethyl cellulose, ethylcarboxyethyl cellulose, polyvinylalcohol, sodium alginate, polyvinylpyrrolidone, vinyl acetate/crotonic acid copolymers, methyl methacrylic ester copolymers, maleic anhydride/methyl vinyl ether copolymers, poly(ethylene oxide), and mixtures thereof.

10. The process according to claim 9, wherein the film-forming polymer is selected from the group consisting of hydroxypropylmethyl cellulose, hydroxypropyl cellulose, and poly(ethylene oxide).

11. The process according to claim 10, wherein the film-forming polymer is hydroxypropylmethyl cellulose.

12. The process according to claim 2, wherein the film-forming polymer is present in an amount of from about 1 wt. % to about 30 wt. %, based on the total weight of the premix of Step (a), which is used to form the wet granulation.

13. The process according to claim 12, wherein the film-forming polymer is present in an amount of from about 3 wt. % to about 7 wt. %.

14. The process according to claim 1, wherein the coating in Step (d) comprises a film-forming polymer.

15. The process according to claim 14, wherein the coating additionally comprises a plasticizer.

16. The process according to claim 15, wherein the plasticizer is selected from the group consisting of acetyl-triethyl citrate, acetyl tributyl-, tributyl-, triethyl-citrate, glycerol diacetate, glycerol triacetate, acetylated monoglycerides, castor oil, dibutyl-phthalate, diamyl-phthalate, diethyl-phthalate, dimethyl-phthalate, dipropyl-phthalate, di-(2-methoxy- or 2-ethoxyethyl)-phthalate, ethylphthalyl glycolate, butylphthalylethyl glycolate, butylglycolate, propylene glycol, polyethylene glycol, diethyladipate, di-(2-methoxy- or 2-ethoxyethyl)-adipate, benzophenone, diethyl- and diburylsebacate, dibutylsuccinate, dibutyltartrate, diethylene glycol dipropionate, ethyleneglycol diacetate, ethyleneglycol dibutyrate, ethyleneglycol dipropionate, tributyl phosphate, tributyrin, PEG sorbitan monooleate, sorbitan monooleate and mixtures thereof.

17. The process according to claim 1, wherein the coating in Step (d) is present in an amount of from about 1 wt. % to about 30 wt. %, based on the total weight of the uncoated granules.

18. The process according to claim 17, wherein the coating is present in an amount of from about 3 wt. % to about 7 wt. %.

19. A pharmaceutical tablet comprising granules which comprise a valproate compound and at least one excipient, wherein said granules are essentially free of an organic solvent.

20. A pharmaceutical tablet comprising granules which comprise a valproate compound and at least one excipient, wherein said granules are coated with a film-forming polymer, and said granules are essentially free of an organic solvent.
